(19) United States
(12) Patent Application Publication
(10) Pub. No.: US 2021/0268146 A1
(43) Pub. Date: Sep. 2, 2021

(54) COATING FOR IMPLANTABLE MEDICAL DEVICES, METHOD FOR PRODUCING SAME, AND IMPLANTABLE MEDICAL DEVICE

(71) Applicant: FRAUNHOFER-GESELLSCHAFT ZUR FÖRDERUNG DER ANGEWANDTEN FORSCHUNG E. V., München (DE)

(72) Inventors: Sascha BALAKIN, Dresden (DE); Jörg OPITZ, Dresden (DE)

(73) Assignee: FRAUNHOFER-GESELLSCHAFT ZUR FÖRDERUNG DER ANGEWANDTEN FORSCHUNG E. V., München (DE)

(21) Appl. No.: 17/259,177

(22) PCT Filed: Jun. 14, 2019
(86) PCT No.: PCT/EP2019/065722

§ 371 Date: Jan. 10, 2021

(30) Foreign Application Priority Data
Jul. 11, 2018 (DE) 10 2018 211 526.5

(51) Int. Cl. A61L 27/34; A61L 27/54; A61L 27/58; A61L 31/10; A61L 31/14; A61L 31/16

(52) U.S. Cl. CPC A61L 27/34; A61L 27/54; A61L 27/58; A61L 31/10; A61L 2420/08; A61L 31/16; A61L 2400/12; A61L 2420/02; A61L 31/148

(57) ABSTRACT

The invention relates to a coating for implantable medical devices. The coating contains a layer complex which comprises at least one polyelectrolyte multilayer and at least one layer of functionalized nanodiamonds. The at least one polyelectrolyte multilayer consists of polyelectrolyte monolayers which are charged positively and negatively in an alternating manner. Additionally, the functionalized nanodiamonds are functionalized with at least one bioactive agent. The invention additionally relates to a method for producing such a coating for implantable medical devices, to the use of such a coating, and to an implantable medical device which has such a coating.

The present invention relates to a coating for implantable medical devices. The coating comprises a layer complex which comprises at least one polyelectrolyte multilayer and also at least one layer of functionalised nanodiamonds. The at least one polyelectrolyte multilayer thereby consists of alternately positively and negatively charged polyelectrolyte monolayers. In addition, the functionalised nanodiamonds are functionalised with at least one bioactive agent. The invention relates in addition to a method for the production of such a coating for implantable medical devices, the use of such a coating and also an implantable medical device which has such a coating.

Implantable medical devices help in the therapy of patients by supporting for example damaged organs or taking over the function thereof. According to the state of the art, such implantable medical devices can be coated with active substances. A fundamental problem with such known coatings is that the output of the active substance is effected in a very uncontrolled manner. Such an uncontrolled output leads however to a (local) overdose of the active substances in the patient and in addition to an uneconomical use of the active substances.

Furthermore, it is known that a polymer blend is applied at the same time as the active substances on the implant surface via spraying or immersion coating. An example is the polymer-based, drug-releasing Stent Eluvia™ (Boston Scientific) for the treatment and restoration of the blood flow of the peripheral arteries above the knee, in particular the Arteria femoralis superficialis (SFA) and the proximal Arteria poplitea (PPA). The Eluvia Stent system is coated with the antirestenotic drug Paclitaxel in conjunction with a polymer.

One disadvantage of such coatings is the continuous output of active substance which is effected generally within 30-60 days. This is only sensible with stents, however not applicable in the case of patient-/disease-specific active substances or antibiotics, growth factors etc.

Starting herefrom, it was the object of the present invention to indicate a coating for implantable medical devices which enables a controlled and/or temporally discrete output of a bioactive agent contained in the coating and also the adjustment of antimicrobial properties of the surface of the implantable medical device.

This object is achieved, with respect to a coating for implantable medical devices, according to patent claim 1, with respect to a method for the production of such a coating, according to patent claim 9 and, with respect to an implantable medical device, by the features of patent claim 15. Patent claim 16 indicates possibilities of use of the coating according to the invention. The respectively dependent patent claims thereby represent advantageous developments.

According to the invention, a coating for implantable medical devices is hence indicated. The coating comprises a layer complex which comprises at least one polyelectrolyte multilayer and also at least one layer of functionalised nanodiamonds. The at least one polyelectrolyte multilayer consists of alternately positively and negatively charged polyelectrolyte monolayers. In addition, the functionalised nanodiamonds are functionalised with at least one bioactive agent.

The polyelectrolyte multilayer can also be termed “polyelectrolyte multilayer (PEM) film”.

The at least one polyelectrolyte multilayer consists of polyelectrolyte monolayers. The polyelectrolyte multilayer thereby comprises at least one positively charged polyelectrolyte monolayer and at least one negatively charged polyelectrolyte monolayer. The polyelectrolyte monolayers are now disposed alternately with respect to their charge. This means that, in the at least one polyelectrolyte multilayer, alternately a positively charged polyelectrolyte monolayer and a negatively charged polyelectrolyte monolayer are always disposed in succession.

Preferably, the layer complex consists of at least one polyelectrolyte multilayer and at least one layer of functionalised nanodiamonds. Preferably, the at least one layer of functionalised nanodiamonds is disposed or deposited on the at least one polyelectrolyte multilayer. The layer complex can comprise one or more polyelectrolyte multilayers and comprise one or more layers of functionalised nanodiamonds. If the layer complex comprises a plurality of polyelectrolyte multilayers and/or a plurality of layers of functionalised nanodiamonds or if the layer complex consists of a plurality of polyelectrolyte multilayers and/or a plurality of layers of functionalised nanodiamonds, the different layers are disposed preferably alternating, i.e., in succession.

The layer complex can hence comprise one or more bi-layers or consist thereof, each of the bi-layers comprising a polyelectrolyte multilayer and a layer of functionalised nanodiamonds or consisting thereof.

Nanodiamonds can also be termed diamond nanoparticles. Nanodiamonds have a size of below 1 μm.

The nanodiamonds are functionalised with at least one bioactive agent, i.e., the at least one bioactive agent is bonded to the nanodiamonds. Such a bonding can be effected for example by physisorption, by electrostatic interactions and/or by covalent interactions.

There is thereby understood by a bioactive agent, a substance which has a biological activity, such as e.g., a pharmaceutical active substance, a biological active substance, a diagnostic active substance, a therapeutic active substance or also mixtures thereof.

By means of the at least one polyelectrolyte multilayer in combination with functionalised nanodiamonds, a particularly advantageous coating for implantable medical devices is obtained. The functionalised nanodiamonds can thereby be immobilised by the polyelectrolyte multilayer on the surface of the implantable medical device, the subsequent output of the bioactive agent from the surface of the implantable medical device being able to be controlled. A controlled or temporally and locally discrete output of the bioactive material is hereby made possible. In addition, the antimicrobial properties of the surface of the implantable medical device can be adjusted thus. Such an adjustment can be effected for example via partial oxidation of the nanodiamonds. As a result, patient tolerance of the implants is increased and the result is faster formation of the functional and structural composite between the organised tissue and the surface of the implant. As a result, the number of revision operations and hence also the total costs can be reduced.

The at least one polyelectrolyte multilayer is self-regulating in the layer thickness and independent of the substrate used and the nanoparticles used (alternating electrical charges). By using the polyelectrolyte multilayer, an immobilisation technique of nanoparticles is hence produced for a broad application. The immobilisation of the nanoparticles is thereby achieved by electrostatic interactions with the polyelectrolyte multilayer. In addition to the nanoparticles, various bioactive agents or active substances and also proteins/peptides can be immobilised. Hence also multifunctional layers with a plurality of active substances can be produced simultaneously. The polyelectrolyte multilayers enable in addition, by the electrostatic interactions, a controlled output of the functionalised nanoparticles, as a result of which also ultimately the output of the bioactive agent can be better controlled.

Polyelectrolyte multilayers can be produced locally on various materials. The result hereof is a high application potential in medical-technological and technological products.

The nanodiamonds offer simple functionalisation strategies (carbon chemistry) and large surfaces for the bonding (e.g., covalent, adsorbed) of bioactive agents or functional groups of such bioactive agents. The bioactive agent can hence be bonded simply to the nanodiamonds and hence integrated in the coating.

After the nanodiamonds, functionalised with the bioactive agent, are released from the coating, a constant and enduring output of the bioactive agent from the surface of the nanodiamonds occurs. The output of the active agent can hence be better controlled and can be effected temporally discretely. This results in increased efficiency with the same quantity of active substances or bioactive agent. As soon as the functionalised nanodiamonds are output into the body, the complex is denatured and the active substance or the bioactive agent develops its effect.

A preferred embodiment of the coating according to the invention is distinguished by the positively charged polyelectrolyte monolayers comprising positively charged polyelectrolytes selected from the group consisting of polydiallyldimethylammonium chloride (PDDA), polyallylamine hydrochloride (PAH), polyethyleneimine (PEI), poly-L-lysine and mixtures hereof or consisting thereof and/or the negatively charged polyelectrolyte monolayers comprising negatively charged polyelectrolytes selected from the group consisting of polystyrene sulphonate (PSS), polyallylamine hydrochloride (PAA), potassium polyvinyl sulphate (PVS), heparin, alginates, pectins, inorganic clay, organic clay and mixtures hereof or consisting thereof.

In a further preferred embodiment of the coating according to the invention, the at least one polyelectrolyte multilayer comprises biodegradable polyelectrolytes or consists thereof. The polyelectrolyte multilayer can thus be constructed in a simple manner and release the functionalised nanoparticles.

A further preferred embodiment of the coating according to the invention is characterised in that the at least one bioactive agent is selected from the group consisting of pharmaceutical active substances, biological active substances, diagnostic active substances, therapeutic active substances and also mixtures hereof.

According to a further preferred embodiment of the coating according to the invention, the layer complex comprises a plurality of polyelectrolyte multilayers and a plurality of layers of functionalised nanodiamonds, the polyelectrolyte multilayers and the layers of functionalised nanodiamonds being disposed alternately. In this way, the output duration of the bioactive agent can be significantly increased. In addition, the temporal course of the output can be better controlled. There is hereby understood by an alternating arrangement that the polyelectrolyte multilayers and the layers of functionalised nanodiamonds are disposed alternately in succession or on each other. The uppermost layer of such a layer complex can concern either a polyelectrolyte multilayer or a layer of functionalised nanodiamonds. The lowermost layer of such a layer complex preferably concerns a polyelectrolyte multilayer.

Furthermore, it is preferred that the polyelectrolyte monolayers have respectively a layer thickness of 5 nm to 5 μm and/or the at least one layer of functionalised nanodiamonds has a layer thickness of 2 nm to 300 nm. The layer thickness of the respective layers can be varied via the field strength of the external electrical field (in the case of an electrophoretic deposition of the layers).

In a further preferred embodiment of the coating according to the invention, the coating has in addition at least one biopolymer layer which comprises at least one biopolymer or consists thereof, which is selected from the group consisting of biopolyesters, proteins, polysaccharides and mixtures hereof. By means of this additional biopolymer layer, the output of the nanodiamonds can be further influenced and controlled. Preferably, the layer complex is disposed on the at least one biopolymer layer.

In addition, it is preferred that the at least one biopolymer layer comprises at least two biopolymer layers, the layer complex being disposed between the at least two biopolymer layers. In other words, the layer complex hereby represents an intermediate layer of a conventional coating.

A further preferred embodiment of the coating according to the invention is distinguished by the coating having in addition a cover layer, the material of the cover layer preferably being selected from the group consisting of
synthetic polymers, preferably polyglycolic acid, polylactide, polycaprolactone, poly(lactide-co-glycolide),
natural polymers, preferably chitin, chitosan, alginates, collagens, gelatines, aerogels, and
mixtures hereof.

The cover layer hereby acts for example as protective layer which protects the further layers from damage. The cover layer represents the outermost or the uppermost layer of the coating.

The present invention relates also to a method for the production of a coating according to the invention on an implantable medical device, in which a layer complex is deposited on an implantable medical device, by
a) at least one polyelectrolyte multilayer which consists of alternately positively and negatively charged polyelectrolyte monolayers being deposited on the implantable medical device or on a layer deposited on the implantable medical device, and
b) at least one layer of nanodiamonds functionalised with at least one bioactive agent being deposited on the polyelectrolyte multilayer.

It is preferred that, after step b), the step sequence of steps a) and b) is repeated at least once. In other words, first step a) and step b) are implemented and subsequently at least once again step a) and step b) are implemented. In this way, a layer complex can be obtained which comprises a plurality of polyelectrolyte multilayers and a plurality of layers of functionalised nanodiamonds, the different layers being disposed alternately, i.e., in succession.

A further preferred variant of the method according to the invention is characterised in that the deposition of the at least one polyelectrolyte multilayer in step a) is effected by alternately positively charged polyelectrolytes as positively charged polyelectrolyte monolayer and negatively charged polyelectrolytes as negatively charged polyelectrolyte monolayer being deposited. In this way the alternating or successive arrangement of the positively and negatively charged polyelectrolyte monolayers can be achieved in a simple manner.

According to a further preferred variant of the method according to the invention, the at least one polyelectrolyte layer in step a) and/or the at least one layer of functionalised nanodiamonds in step b) are deposited via electrostatic interactions and/or by means of an external electrical field.

A further preferred variant of the method according to the invention is distinguished in addition by at least one biopolymer layer being deposited, which comprises at least one biopolymer or consists thereof, which is selected from the group consisting of biopolyesters, proteins, polysaccharides and mixtures hereof. Preferably, the biopolymer layer is deposited on the implantable medical device before step a).

In addition, it is preferred that at least two biopolymer layers are deposited, the deposition being effected such that the layer complex is disposed between the at least two biopolymer layers. At least one biopolymer layer can hereby be deposited on the implantable medical device before step a) and at least one further biopolymer layer on the layer complex after step b).

In a further preferred variant of the method according to the invention, in addition at least one cover layer is deposited, the material of the cover layer being selected from the group consisting of
synthetic polymers, preferably polyglycolic acid, polylactide, polycaprolactone, poly(lactide-co-glycolide),
natural polymers, preferably chitin, chitosan, alginates, collagens, gelatines, aerogels, and
mixtures hereof.

The cover layer can be deposited on the layer complex or on an additional biopolymer layer, if this is present.

The present invention relates in addition to an implantable medical device which has a coating according to the invention. The layer complex is hereby deposited on the implantable medical device or on a layer deposited on the implantable medical device. In addition to the layer complex, the coating can have for example at least one biopolymer layer and a cover layer, the at least one biopolymer layer being disposed on the surface of the implantable medical device and/or on the layer complex, the layer complex being disposed on the surface of the implantable medical device and/or on the at least one biopolymer layer, and the cover layer being disposed or deposited on the layer complex or on the at least one biopolymer layer. Very particularly preferably, the at least one biopolymer layer is disposed or deposited on the surface of the implantable medical device, the layer complex on the at least one biopolymer layer, and the cover layer on the layer complex.

Furthermore, the present invention relates to the use of a coating according to the invention or of an, according to the invention, implantable medical device for controlled and/or temporally discrete output of a bioactive agent which is selected preferably from the group consisting of pharmaceutical active substances, biological active substances, diagnostic active substances, therapeutic active substances and also mixtures hereof.

The present invention is intended to be explained in more detail on the basis of the subsequent Figures and examples without wishing to restrict said invention to the specific embodiments and parameters shown here.

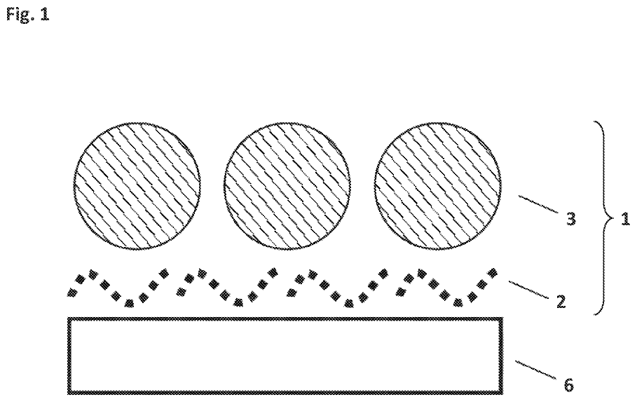

In FIG. 1, a first embodiment, given by way of example, of the coating according to the invention for implantable medical devices is illustrated. This comprises a layer complex 1 which consists of a polyelectrolyte multilayer 2 and also a layer 3 of functionalised nanodiamonds. The polyelectrolyte multilayer 2 thereby consists of alternately positively and negatively charged polyelectrolyte monolayers. In addition, the functionalised nanodiamonds are functionalised with at least one bioactive agent. The coating can be disposed or deposited on a substrate, e.g., an implantable medical device 6.

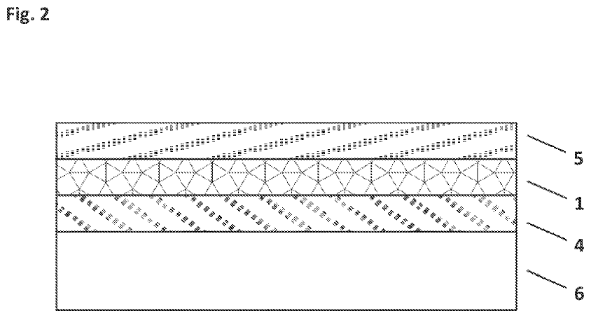

In FIG. 2, a second embodiment, given by way of example, of the coating according to the invention for implantable medical devices is illustrated. The coating here is disposed or deposited on an implantable medical device 6. The coating here comprises a layer complex 1 which consists of a plurality of polyelectrolyte multilayers and also a plurality of layers of functionalised nanodiamonds, these being disposed alternately. In other words, the layer complex 1 comprises a plurality of bi-layers which consist respectively of a polyelectrolyte multilayer and a layer of functionalised nanodiamonds. The polyelectrolyte multilayers and the layers of functionalised nanodiamonds or the precise construction of the layer complex are not shown in FIG. 2 for reasons of clarity. Furthermore, the coating has a biopolymer layer 4 which comprises at least one biopolymer or consists thereof, which is selected from the group consisting of biopolyesters, proteins, polysaccharides and mixtures hereof. The layer complex 1 is disposed or deposited on the biopolymer layer 4, the biopolymer layer 4 being disposed or deposited on the surface of the implantable medical device 6. In addition, the coating has in addition a cover layer 5) which is disposed or deposited on the layer complex 1.

1-16. (canceled)

17. A coating for an implantable medical device, the coating comprising a layer complex which comprises at least one polyelectrolyte multilayer and at least one layer of functionalised nanodiamonds, wherein the at least one polyelectrolyte multilayer consists of alternately positively and negatively charged polyelectrolyte monolayers and the functionalised nanodiamonds are functionalised with at least one bioactive agent.

18. The coating according to claim 17, wherein
the positively charged polyelectrolyte monolayers comprise a positively charged polyelectrolyte selected from the group consisting of polydiallyldimethylammonium chloride, polyallylamine hydrochloride, polyethyleneimine, poly-L-lysine and mixtures thereof, and/or
the negatively charged polyelectrolyte monolayers comprise a negatively charged polyelectrolyte selected from the group consisting of polystyrene sulphonate, polyallylamine hydrochloride, potassium polyvinyl sulphate, heparin, alginates, pectins, inorganic clay, organic clay and mixtures thereof.

19. The coating according to claim 17, wherein the at least one polyelectrolyte multilayer comprises biodegradable polyelectrolytes.

20. The coating according to claim 17, wherein the at least one bioactive agent is selected from the group consisting of pharmaceutical active substances, biological active substances, diagnostic active substances, therapeutic active substances, and mixtures thereof.

21. The coating according to claim 17, wherein the layer complex comprises a plurality of polyelectrolyte multilayers and a plurality of layers of functionalised nanodiamonds, and the polyelectrolyte multilayers and the layers of functionalised nanodiamonds are disposed alternately.

22. The coating according to claim 17, wherein the polyelectrolyte monolayers have respectively a layer thickness of 5 nm to 5 μm and/or the at least one layer of functionalised nanodiamonds has a layer thickness of 2 nm to 300 nm.

23. The coating according to claim 17, wherein the coating has, in addition, at least one biopolymer layer which comprises at least one biopolymer selected from the group consisting of biopolyesters, proteins, polysaccharides and mixtures thereof.

24. The coating according to claim 17, wherein the coating has, in addition, a cover layer.

25. The coating according to claim 24, wherein the material of the cover layer is selected from the group consisting of synthetic polymers, natural polymers, and mixtures thereof.

26. A method for producing a coating according to claim 17, on an implantable medical device, in which a layer complex is deposited on an implantable medical device, the method comprising the steps of:
(a) depositing at least one polyelectrolyte multilayer which consists of alternately positively and negatively charged polyelectrolyte monolayers deposited on the implantable medical device or on a layer deposited on the implantable medical device, and
(b) depositing at least one layer of nanodiamonds functionalised with at least one bioactive agent on the polyelectrolyte multilayer.

27. The method according to claim 26, wherein after step (b), the step sequence of steps (a) and (b) is repeated at least once.

28. The method according to claim 26, wherein the deposition of the at least one polyelectrolyte multilayer in step (a) is effected by depositing alternately positively charged polyelectrolytes as positively charged polyelectrolyte monolayer and negatively charged polyelectrolytes as negatively charged polyelectrolyte monolayer.

29. The method according to claim 26, wherein the at least one polyelectrolyte multilayer in step (a) and/or the at least one layer of functionalised nanodiamonds in step (b) are deposited via electrostatic interactions and/or by means of an external electrical field.

30. The method according to claim 26, which further comprises depositing at least one biopolymer layer, wherein the biopolymer is selected from the group consisting of biopolyesters, proteins, polysaccharides, and mixtures thereof.

31. The method according to claim 30, wherein the biopolymer layer is deposited on the implantable medical device before step (a).

32. The method according to claim 26, further comprising depositing at least one cover layer, wherein the material of the cover layer is selected from the group consisting of synthetic polymers, natural polymers, and mixtures thereof.

32. The method according to claim 32, wherein the synthetic polymer is selected from the group consisting of polyglycolic acid, polylactide, polycaprolactone, and poly(lactide-co-glycolide), and mixtures thereof; and/or the natural polymers are selected from the group consisting of chitin, chitosan, alginates, collagens, gelatins, aerogels, and mixtures thereof.

33. An implantable medical device which has a coating according to claim 17.

34. A method of providing controlled and/or temporally discrete output of a bioactive agent comprising utilizing a medical device according to claim 33.

35. The method according to claim 34, wherein the bioactive agent is selected from the group consisting of pharmaceutical active substances, biological active substances, diagnostic active substances, therapeutic active substances, and mixtures thereof.
